(19) United States
(12) Patent Application Publication
(10) Pub. No.: US 2026/0224131 A1
(43) Pub. Date: Aug. 6, 2026

(54) Patent Documentation for an AI-Powered System and Method for Remote Monitoring and Assessment of Joint Health Using Image and Video Analysis

(71) Applicant: Nick Hughes, Hertfordshire (GB)

(72) Inventor: Nick Hughes, Hertfordshire (GB)

(21) Appl. No.: 19/044,682

(22) Filed: Feb. 4, 2025

(51) Int. Cl. A61B 5/11; A61B 5/00; G16H 50/20; G16H 70/20

(52) U.S. Cl. CPC A61B 5/1128; A61B 5/1121; A61B 5/445; A61B 5/7267; G16H 50/20; G16H 70/20

(57) ABSTRACT

The AI-powered system for monitoring joint health is an innovative software-based medical device designed to transform arthritis care by addressing the limitations of traditional methods. Unlike conventional approaches that rely on subjective clinical evaluations and lengthy in-person consultations, this system utilises AI-driven algorithms to deliver real-time, objective assessments. Its unique ability to combine patient-reported outcomes with precise motion analysis sets a new standard for accuracy and efficiency in arthritis management. Utilising cameras on smartphones, tablets, or IoT devices, the system captures video frames or static images portraying a patient's joints, which are analysed by advanced machine learning algorithms. These algorithms detect joint positions, joint angles, range of motion, swelling, and skin discolouration, providing a comprehensive assessment of disease indicators. This comprehensive evaluation is achieved through machine learning models trained on extensive, clinically validated datasets. By leveraging these datasets, the system ensures high accuracy and reliability in its analysis, offering robust insights into the patient's condition. The system integrates this objective data with patient-reported outcomes to calculate a Digital Disease Activity Score (DDAS) that mirrors clinical standards like DAS-28, a benchmark recognised for its accuracy in assessing arthritis severity. By aligning with DAS-28, the system ensures compatibility with established clinical practices and enhances its utility in guiding treatment decisions. This integration supports precise monitoring of disease progression and facilitates personalised care recommendations, ultimately improving patient outcomes. Equipped with a user-friendly interface and secure cloud integration, the platform ensures compliance with data protection regulations while facilitating seamless data sharing between patients and clinicians. The interface provides intuitive navigation, clear instructions, and multilingual support, enhancing accessibility for diverse patient groups. Secure cloud integration adheres to stringent standards such as GDPR, DPA, and HIPAA, safeguarding sensitive patient data through robust encryption protocols and secure data transfer mechanisms. By enhancing accuracy, scalability, and accessibility, the system significantly improves patient outcomes and reduces healthcare burdens in arthritis management. This system is an assistive monitoring tool, not a diagnostic device, and is intended to provide objective assessments to support clinical decision-making rather than replace formal medical evaluations.

### TECHNICAL DESCRIPTION OF INVENTION

The invention is an Artificial Intelligence (AI)-powered system designed to remotely monitor and assess joint health using video and image data captured from any camera-equipped device, including but not limited to smartphones, tablets, IoT devices, wearable devices, or any future visual capture apparatus capable of capturing joint movements. The system is hardware-agnostic and can be implemented on any device with an internet browser and camera, ensuring broad applicability and scalability across diverse healthcare settings. The system employs advanced machine learning algorithms to analyse joint range of motion (ROM), detect visual indicators of arthritis (e.g., swelling, skin discolouration), and generate a Digital Disease Activity Score (DDAS). This score provides a comprehensive assessment of disease activity, enabling early intervention and personalised treatment strategies. The system integrates seamlessly into clinical workflows, offering real-time data for healthcare providers and empowering patients to manage their condition from home. This system is an assistive monitoring tool, not a diagnostic device, and is intended to provide objective assessments to support clinical decision-making rather than replace formal medical evaluations.

### BACKGROUND OF THE INVENTION

Arthritis, a widespread and debilitating condition, impairs joint mobility and causes persistent pain, affecting millions worldwide and diminishing their quality of life. Traditional methods of assessing arthritis often rely on subjective clinical evaluations, which are resource-intensive and generally require physical patient presence. These conventional approaches usually struggle to deliver timely and precise insights, barriers that hinder effective disease management and place a heavy burden on healthcare systems globally.

The invention of an AI-powered system for joint health monitoring offers a revolutionary solution to these challenges by providing a highly accessible, objective, and data-driven alternative to traditional methods. Unlike conventional approaches that rely heavily on subjective evaluations and physical presence, this system utilises advanced AI algorithms to deliver precise and timely insights into joint health. This transformative capability significantly enhances the efficiency and accuracy of arthritis care. By leveraging any camera-equipped device, including but not limited to smartphones, tablets, IoT devices, wearable devices, or any future visual capture apparatus, the system facilitates remote assessments of joint conditions. This hardware-agnostic approach ensures that the system can be widely adopted and adapted to new technologies as they emerge. Utilising advanced computer vision and machine-learning (ML) technologies, it captures images and video of joint movements, which are processed through utilising advanced computer vision techniques, such as Convolutional Neural Networks (CNNs), the system captures and analyses images and videos of joint movements. These models have demonstrated accuracy within 1 degree when validated against human-annotated datasets. These algorithms can analyse data at high speeds, providing precise measurements and actionable insights into joint health in real-time. These algorithms accurately measure the range of motion (ROM) and generate a Digital Disease Activity Score (DDAS), providing critical insights into the patient's arthritic condition. The precise ROM measurements enable clinicians to identify joint abnormalities and track progression over time, while the DDAS integrates this objective data with patient-reported outcomes to support tailored treatment strategies and evidence-based clinical decision-making.

The AI models employed by the system detect critical indicators like joint swelling and skin discolouration, which are associated with varying degrees of arthritis severity. This process allows for consistent remote monitoring and data analysis, producing reliable outcomes without the need for constant in-person evaluations. The accessibility, objectivity, and scalability of the system not only make it a promising tool for transforming arthritis care but also have the potential to significantly lower healthcare costs while improving patient outcomes.

### SUMMARY OF INVENTION

The proposed invention, the Rest Health AI-powered system, substantively addresses these challenges by providing a scalable and objective alternative to traditional methods, which often rely on subjective evaluations and in-person consultations. It streamlines joint health monitoring with advanced AI algorithms, offering precision and efficiency not achievable through conventional approaches. It employs sophisticated computer vision technology in conjunction with proprietary, Python-based machine learning algorithms. This system facilitates the remote and continuous monitoring of joint health utilising any camera-equipped device, including but not limited to smartphones, tablets, Internet of Things (IoT) devices, wearable devices, or any future visual capture apparatus capable of capturing joint movements. The system is designed to be hardware-agnostic, ensuring compatibility with a wide range of devices and future technological advancements. The core functionality involves the acquisition of video frames or static images depicting a patient's joints either in a static position (e.g., resting their hand on a table to capture their fingers, hand, and wrist) or performing a simple action (e.g., sitting on a chair, lifting their arms above their head, and then returning to a resting position). This process allows for the assessment of the patient's motion. Subsequently, this data is processed through bespoke algorithms designed to precisely measure the patient's range of motion (ROM) of their joints, delivering measurements with exceptional accuracy to within 1 degree.

Furthermore, the system computes a Digital Disease Activity Score (DDAS) by integrating data from visual inputs provided by the patient. Enhanced by AI models capable of recognising conditions signified by visual markers-such as joint swelling or skin discolouration-associated with arthritic pathologies, the system ensures reliability and consistency in the insights it provides. The system's AI models are trained on extensive datasets and validated against clinical standards, ensuring high accuracy and reliability. The platform also features a user-friendly interface, secure cloud integration, and compliance with data protection regulations (e.g., GDPR, DPA, HIPAA), making it accessible and scalable for widespread adoption in healthcare settings. When accessed via a web browser, the system securely transmits data to cloud servers for remote processing, ensuring both performance efficiency and compliance with industry standards for data handling. To uphold strict data security measures and regulatory compliance (GDPR, DPA, HIPAA), the system integrates AES-256 encryption for data storage and TLS 1.3 encryption for secure transmission, safeguarding sensitive medical information. Furthermore, patient data undergoes pseudonymisation to protect privacy while preserving analytical value, ensuring that de-identified records remain useful for research and clinical applications. This innovative approach represents a significant advancement in arthritis care, offering potential cost reductions to healthcare systems and improvements in patient outcomes.

The AI-powered system for monitoring joint health is an advanced medical solution designed to overcome the limitations of traditional arthritis assessments. By leveraging cameras on smartphones, tablets, or IoT devices, the system captures joint movements and static images during predefined exercises that ensure clinical consistency. The system's AI Analysis Engine, developed in Python, processes the captured data using a combination of convolutional neural networks (CNNs) and random forest machine learning models. CNNs are employed for precise joint position capture and swelling identification by analysing spatial patterns in image and video data. In parallel, random forest models classify and detect joint discolouration by evaluating colour variations in the pixel data extracted from joint areas. These models operate within a unified pipeline, where the CNNs first extract relevant joint features, and the random forest classifiers then interpret additional visual markers to enhance the overall assessment accuracy.

To enable real-time processing, the system incorporates edge computing techniques, allowing video and image data to be processed locally on the device when using the downloadable app. This ensures low-latency analysis and immediate feedback for both patients and clinicians. When accessed via a web browser, the system securely transmits data to cloud servers for remote processing, ensuring scalability and consistent performance across diverse platforms. The training process relies on supervised learning with extensively labelled clinical datasets, ensuring high precision in detecting joint angles, range of motion (ROM), and visual indicators of arthritis. By integrating advanced AI methodologies with real-time processing capabilities, this system provides an innovative, scalable, and clinically validated solution for remote joint health monitoring. This system's proprietary algorithms detect joint angles, range of motion (ROM), as well as visual indicators like swelling and skin discolouration that may signal various arthritic conditions. By analysing individual video frames, the AI ensures accurate and reliable scoring of joint health, providing a detailed understanding of each patient's condition.

A key feature of the system is the Digital Disease Activity Score (DDAS), which integrates objective data with patient-reported outcomes such as pain, functionality, and swelling. This integration mirrors the DAS-28 standard used in clinical practices, combining data with individually tailored thresholds from pose estimation models for precise detection of joint anomalies. The detailed DDAS calculation empowers clinicians and patients alike, offering insights into potential flare-ups, disease progression, and guiding personalised treatment strategies.

An intuitive user interface facilitates patient assessments, providing clear instructions and enabling clinicians to access analytic dashboards for both real-time and longitudinal monitoring. Data handling is enhanced through secure cloud integration, ensuring compliance with data protection regulations like GDPR, DPA and HIPAA, and facilitating seamless communication among patients and healthcare providers.

The system is equipped with features such as progress tracking, flare-up reports, and seamless integration with electronic health records (EHR), which significantly improve clinical workflows by reducing administrative burdens and enabling real-time data access. These features enhance patient care outcomes by providing clinicians with actionable insights for timely decision-making and supporting personalised treatment plans. Its adaptable AI architecture allows for continuous improvement and customisation as new data becomes available. The system utilises robust testing frameworks to validate AI model accuracy against pre labelled datasets, ensuring reliability and compliance with clinical standards. For example, the system refines its machine learning models based on additional patient data, incorporates updates to clinical guidelines, and dynamically adjusts clinically validated thresholds for improved joint health metrics to enhance accuracy and applicability across diverse patient populations.

### DESCRIPTION OF DRAWINGS

FIG. 1 illustrates the overall architecture of the AI-powered system, detailing the flow of data from capture to processing and presentation of insights.

FIG. 2 illustrates the continuous improvement process of the AI architecture, detailing how data collection, processing, model updates, and deployment work together to enhance the system's accuracy and adaptability

FIG. 3 showcases the user interfaces used for patient data collection and results display.

FIG. 4 illustrates the AI-driven analysis of joint angles and range of motion (ROM) feature used to assess patient mobility.

FIG. 5 illustrates the step-by-step process for calculating the Digital Disease Activity Score (DDAS) by integrating AI-driven joint analysis with patient-reported data.

FIG. 6 highlights the security architecture of the system, detailing the process of secure data capture, encryption, processing, and controlled access for patient and clinician use.

### DETAILED DESCRIPTION OF INVENTION

At the heart of the system is the AI Analysis Engine. Developed in Python, this engine leverages cutting-edge machine learning algorithms, including convolutional neural networks (CNNs), and random forest models trained on comprehensive datasets comprising thousands of annotated joint datasets, images and videos of joint movements. The random forest models play a key role in enhancing classification tasks by combining multiple decision trees to improve prediction accuracy and robustness. The training process involves supervised learning, where the models are trained to detect joint angles, range of motion (ROM), and visual indicators of arthritis (e.g., swelling, skin discolouration) using labelled data from clinical studies. The datasets are continuously updated with new patient data to improve model accuracy and adaptability. The purpose of this engine is to scrutinise the captured video frames or images to identify and quantify joint angles, range of motion (ROM), and visual indicators such as swelling and skin discolouration, all critical markers of arthritic conditions. This AI-driven analysis is modular, allowing for the examination of individual frames to ensure precision and reliability across each evaluation. With an emphasis on high-quality data analysis, the engine achieves unparalleled accuracy in detecting nuanced joint movements and disease indicators, thus providing invaluable insights into the patient's condition.

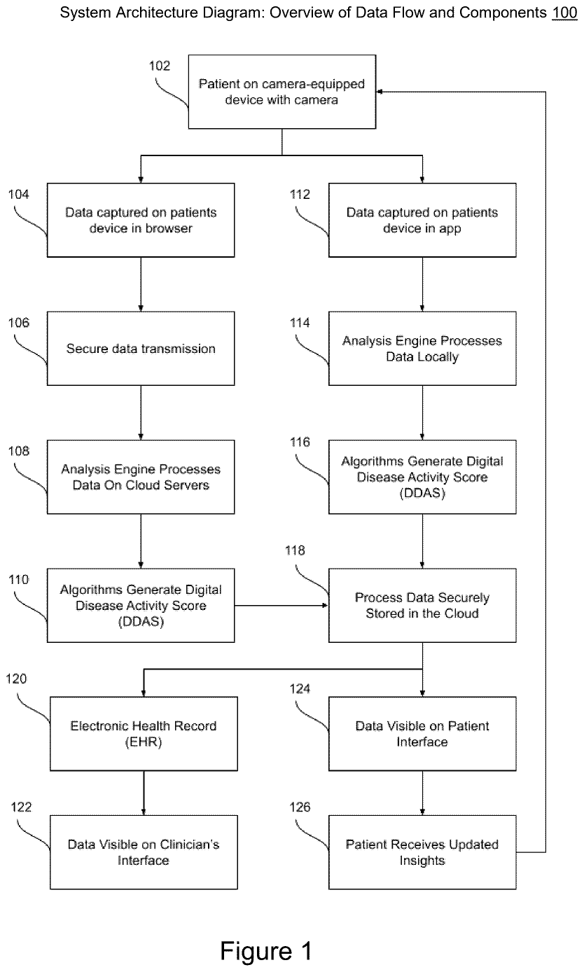

FIG. 1 illustrates the overall architecture of the AI-powered system, detailing the flow of data from capture to processing and presentation of insights. The process begins with a patient using a camera-equipped device 102 such as a smartphone, tablet, or IoT device to capture joint movement data. If the patient accesses the system through a web browser, the captured data 104 is securely transmitted 106 to cloud servers for further analysis. When using the dedicated app, data 112 is processed locally 114 before being stored securely in the cloud 118.

Once received, the AI Analysis Engine processes the data either on cloud servers 108 or locally on the device 114, depending on the mode of access. The AI models extract movement metrics from images and videos, identifying key joint positions, angles, and abnormalities, such as discolouration or swelling. The extracted data is then processed by in-house Python algorithms, which compute the Digital Disease Activity Score (DDAS) 110, 116 using predefined clinical thresholds and weighted computations.

The computed DDAS and associated insights are securely stored 118 and integrated with Electronic Health Records (EHR) 120 for clinician access. Processed results are displayed on both clinician and patient interfaces. Clinicians can review computed insights and DDAS scores via their dedicated dashboard 122, while patients can access their processed results, recommendations, and real-time feedback through the patient-facing application 124. Finally, patients receive updated insights 126, enabling them to monitor disease progression and make informed decisions about their treatment.

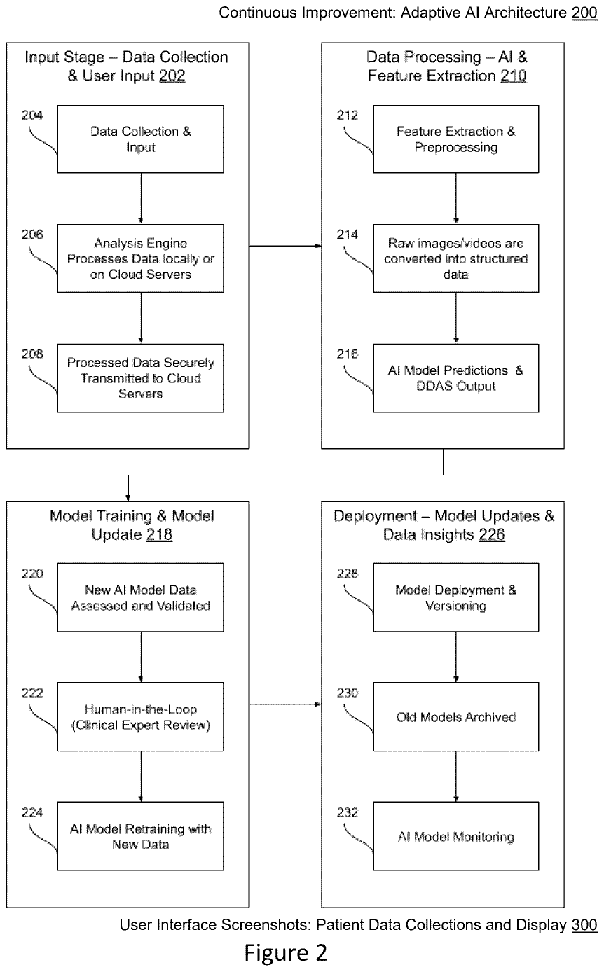

FIG. 2 illustrates the continuous improvement process of the AI architecture, detailing how data collection, processing, model updates, and deployment work together to enhance the system's accuracy and adaptability. The process begins with the input stage 202, where patient data is collected 204 and processed locally or on cloud servers 206 before being securely transmitted for further analysis 208.

In the data processing and feature extraction stage 210, raw images and videos are pre-processed 212, and AI models extract key movement metrics, converting them into structured data 214. This extracted data is then fed into the AI model to assist in Digital Disease Activity Score (DDAS) Output predictions 216, which are used for clinical insights.

The system continuously updates itself through model training and retraining 218. New AI model data is assessed and validated 220, incorporating human-in-the-loop clinical expert review 222 to validate and adjust AI-generated predictions based on expert feedback, ensuring alignment with clinical standards. These validated adjustments are then used to retrain the model, improving accuracy and reliability over time. The model undergoes retraining with newly labelled datasets 224 to ensure improved predictions.

Deployment and model updates 226 ensure that the system remains accurate and up-to-date. New models are deployed and versioned 228, while older models are archived 230. AI model monitoring 232 enables ongoing evaluation, ensuring that the deployed models function correctly and adapt to new patient data over time. FIG. 2 depicts the adaptive AI architecture, showing components such as data input from patients 200, retraining of models with updated datasets, validation of model accuracy, and deployment of improved algorithms.

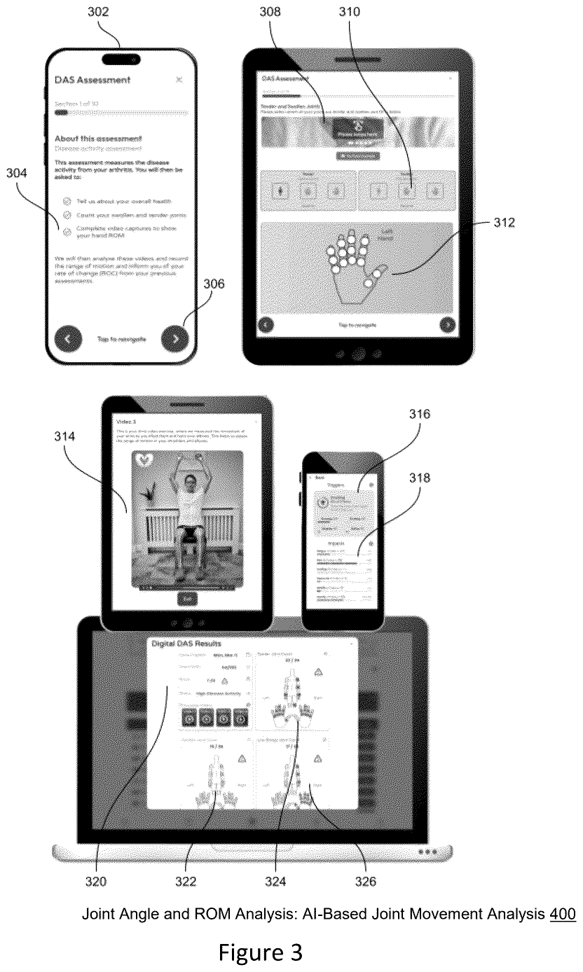

FIG. 3 showcases the user interfaces used for patient data collection and result display. The patient-facing application 302 is shown on a smartphone, where users can start a DDAS assessment and are given an overview of the information required on overall health, such as joint pain, functional ability, tenderness, swelling, and range of motion. The interface 304 provides structured lists to inform data entry, while interactive, easy-to-use navigation 306 allows seamless progression through the assessment. All text-based content in the interface is designed for multilingual support, ensuring accessibility for users across different regions and languages.

A tablet-based interface 308 displays a detailed assessment screen where users receive prompts that can be translated into multiple languages. The language is automatically set based on the user's profile settings, ensuring accessibility for diverse users and improving usability across different regions. Users can input tenderness and swelling indicators 310 by selecting areas on a body or hand diagram. Below this, a visual representation 312 dynamically updates to show the affected joints selected by the user. These selections are stored by the system and integrated into the Digital Disease Activity Score (DDAS) calculation, allowing for a more personalised and data-driven assessment of disease activity.

Another tablet interface example 314 displays the processed output of a patient's recorded exercise. The system overlays a wireframe representation of detected joint movements onto the original video, allowing users to visually compare their movements with AI-extracted joint positions providing confidence in the systems output. This feature helps patients understand their range of motion and highlights any deviations from expected movement patterns, providing valuable insight into their condition.

A mobile interface 316 displays real-time disease activity tracking, with patient-specific triggers and symptom trends visible in a structured dashboard 318. The DDAS assessment output 320, displayed on a laptop, also scalable to a tablet or smaller mobile device presents comprehensive DDAS results. The interface includes a detailed breakdown of the Digital Disease Activity Score (DDAS), highlighting swollen joints selected by the user 322, tender joints selected by the user 324, and joints that do not exhibit a range of motion within healthy thresholds 326. This multi-device interface design ensures accessibility and streamlined patient-clinician interaction across different platforms, with text-based outputs that can be translated into multiple languages for improved usability.

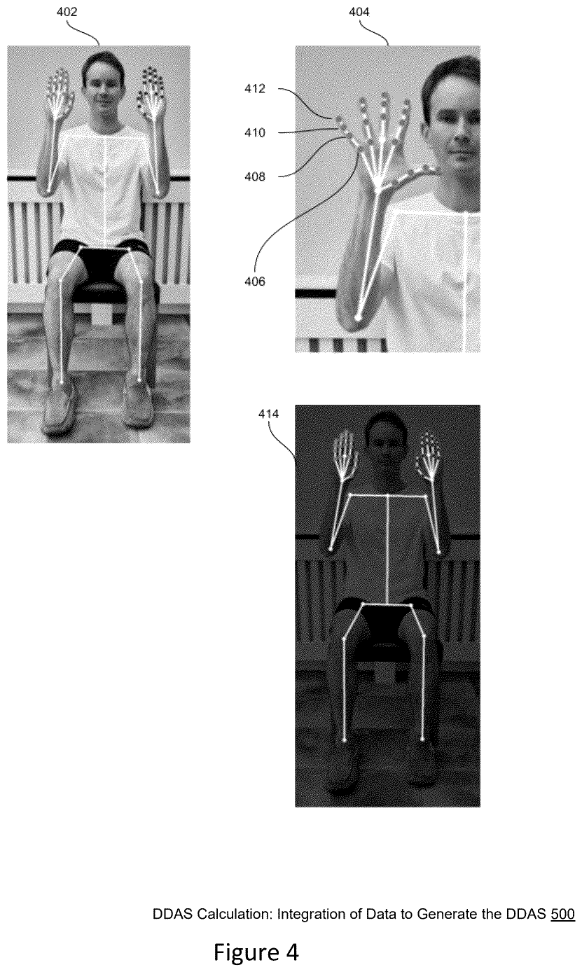

FIG. 4 illustrates the AI-driven analysis of joint angles and range of motion (ROM) feature used to assess patient mobility. The first image 402 presents an example of Exercise Video 1 of 4, which can be performed while sitting or standing. This exercise focuses on measuring the range of motion in the wrists and fingers. In 402, the system captures the initial position with hands open and fingers closed, ensuring that wrist alignment and overall hand posture are recorded accurately.

The second image from twenty-five frames later in the video or one second later, 404 displays the next position, where the patient fully opens their fingers. This movement allows the system to measure the range of motion in the metacarpophalangeal (MCP) joints 406. The AI-based pose estimation model uses the wrist position as a reference, tracking movement across the MCP joint and the proximal interphalangeal (PIP) joint 408. The distal interphalangeal (DIP) joints and fingertip (TIP) positions are also identified but are not actively used in this assessment.

To calculate a joint angle, the system uses three key points, in this case: the wrist, the little finger's MCP, and the little finger's PIP joints. The angle is determined using the vector-based angle calculation (VBAC) to compute the difference in vector slopes:

$$angle = \vert VBAC ( y\_c - y\_b , x\_c - x\_b ) - VBAC ( y\_a - y\_b , x\_a - x\_b ) \vert$$

where:
(x_a, y_a) represents the wrist position in its x and y position,
(x_b, y_b) represents the MCP joint in its x and y position (midpoint),
(x_c, y_c) represents the PIP joint in its x and y position.

The computed angle is then converted from radians to degrees. At rest, the joint is considered to be at 0 degrees, with movement measured from this neutral position. The system calculates angles ranging from 0 to +90 degrees for abduction and 0 to −90 degrees for adduction, ensuring an accurate representation of joint motion within these thresholds.

The final processed output 414 serves as a test image in low-light conditions, with 75% of the brightness removed to assess the model's robustness. Despite the reduced brightness, the model maintains high accuracy, correctly identifying joint positions within one degree of their actual location. The system only begins to experience a decline in confidence levels when brightness is reduced by 90%, particularly in low-contrast images. This demonstrates the effectiveness of the AI model in challenging conditions and highlights its ability to maintain accuracy even in suboptimal lighting environments.

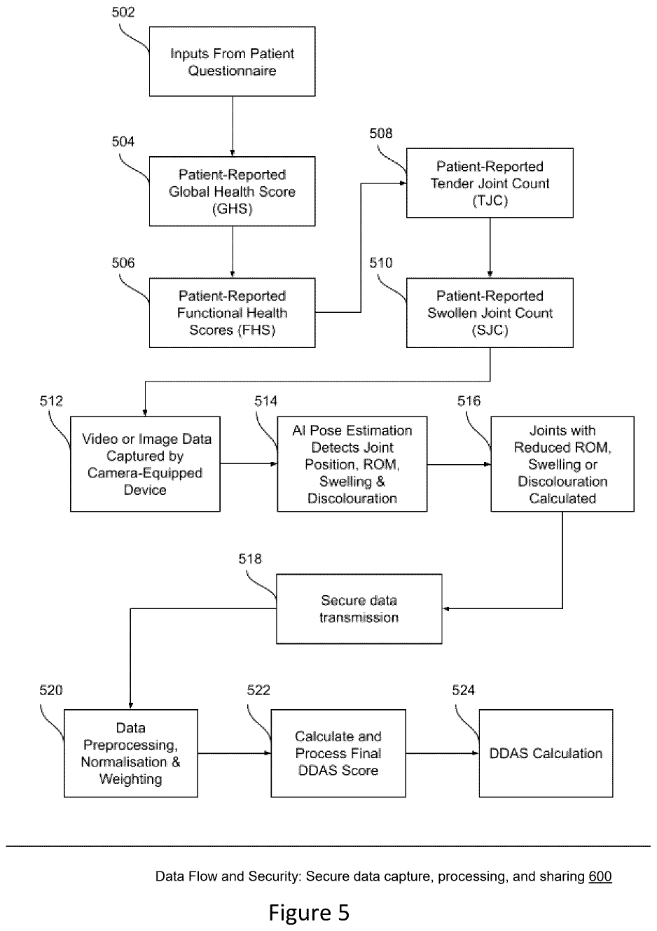

FIG. 5 illustrates the step by-step process for calculating the Digital Disease Activity Score (DDAS) by integrating AI-driven joint analysis with patient-reported data.

The workflow begins with inputs from the patient questionnaire 502, where individuals provide their personal health assessments. This includes global health score (GHS) 504, which ranges from 0 to 100 based on the severity of arthritis symptoms, and functional health scores (FHS) 506, which assess mobility through exercises involving motion, gripping, and twisting, scoring all these individually from 0 to 5. Additionally, tender joint count (TJC) 508 and swollen joint count (SJC) 510 allow patients to self-assess and report joint tenderness and swelling, contributing to a comprehensive evaluation of disease activity.

Next, our AI captures objective data through video or image input from a camera-equipped device 512. The AI-driven pose estimation model 514 detects joint positions in each frame or image, enabling our algorithms to compute the patient's joint angles with high precision. As each joint position is scanned, the system also checks for discolouration and swelling to identify potential signs of inflammation or disease progression. Once all frames have been processed, the system calculates the range of motion (ROM) by analysing joint movement over time, identifying any limitations or deviations from expected motion or expected visual patterns 516. This allows for a comprehensive assessment of joint mobility and potential restrictions.

The collected data from both the patient-reported inputs and AI analysis is securely transmitted 518 for further processing. The system then applies data preprocessing, normalisation, and weighting 520, ensuring that each parameter is appropriately factored into the final DDAS calculation. The weighted values are then combined using a computational model that accounts for joint impairments, self-reported symptoms, and clinically validated assessment factors.

In the final step, the system calculates and processes the DDAS score 522, integrating all weighted variables to generate an accurate disease activity classification. This final DDAS calculation 524 assigns a disease activity category-remission, low, moderate, or high-allowing clinicians to make data-driven decisions for patient treatment and disease monitoring.

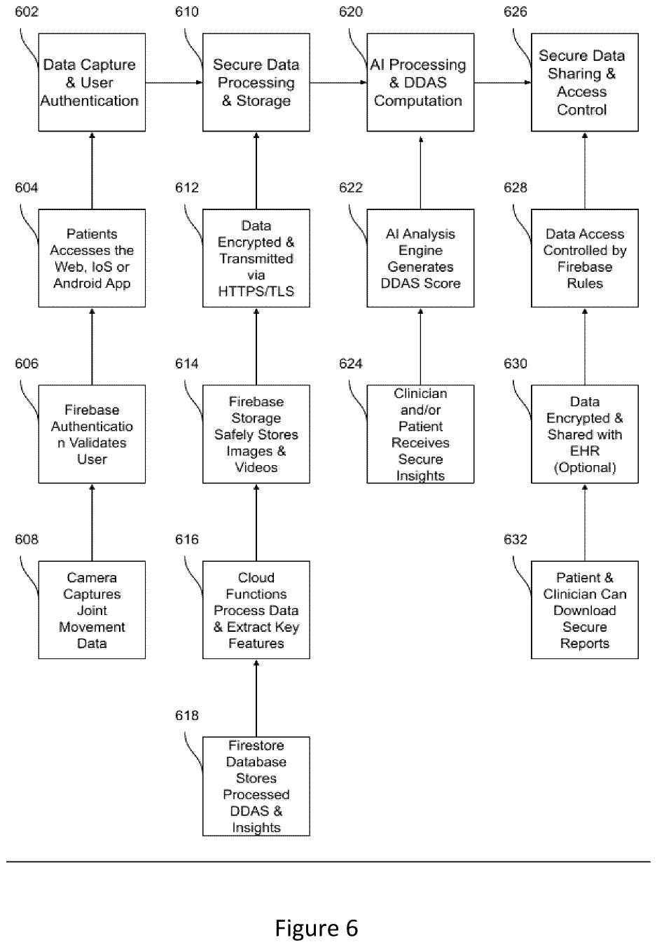

FIG. 6 highlights the security architecture of the system, detailing the process of secure data capture, encryption, processing, and controlled access for patient and clinician use.

The process begins with data capture and user authentication 602. Patients access the system via a web, iOS, or Android app 604, where Firebase Authentication 606 validates user identity, ensuring secure access control. Once authenticated, the system captures joint movement data 608 through the device's camera.

Captured data is then sent for secure data processing and storage 610. Data is encrypted and transmitted via HTTPS/TLS 612, ensuring end-to-end security during transfer. Firebase Storage 614 safely stores images and videos, while Cloud Functions 616 extract key features from the raw data. Processed results, including DDAS scores, are stored in the Firestore database 618.

The AI system processes the stored data in the AI Processing & DDAS Computation phase 620. Here, the AI Analysis Engine 622 generates the DDAS score, which is then securely made available to clinicians and/or patients 624.

Finally, the system ensures secure data sharing and access control 626. Access to stored data is strictly controlled by Firebase rules 628, allowing only authorised users to retrieve information. Optionally, data can be encrypted and shared with EHR systems 630 for seamless integration into clinical workflows. Patients and clinicians can also download secure reports 632 for offline reference or medical documentation.

This security framework ensures that all patient data remains protected, with encrypted transmission, cloud-based storage security, and controlled access policies that align with regulatory standards such as GDPR, HIPAA, and DPA. FIG. 6 highlights secure data capture, transmission, processing, and storage mechanisms 600, including encryption protocols, Firebase Authentication, and integration with external EHR systems.

Central to the system's functionality is the Video Capture Module, which utilises readily available technology-smartphones, tablets, or IoT devices equipped with cameras-to systematically document joint movements, and joint health. The system utilises edge computing techniques to process video and image data locally on the device, when the patient uses the downloadable application, enabling real-time analysis of joint movements with minimal latency. Alternatively, when accessed via a web browser, the system securely transmits data to cloud servers for processing, ensuring scalability and consistent performance across different platforms. This approach ensures that data is processed efficiently, even in low-bandwidth environments, and provides immediate feedback to patients and clinicians. Patients are guided through a series of clinically validated exercises, such as controlled hand movements and specific joint actions like flexion and extension, ensuring that data collection adheres to predefined standards for consistency. These exercises are designed to target critical motion patterns, allowing for the capture of precise and meaningful data for analysis. This process can include capturing static images, which, when in needed combination with video frames, provide a robust foundation for thorough joint assessment. This comprehensive approach replaces the subjective and resource-intensive traditional evaluations, which often rely on manual measurements and in-person consultations, with a more precise, scalable solution. Traditional methods are prone to variability between clinicians and delayed insights due to logistical constraints, whereas this system ensures consistent, objective analysis through AI-driven automation, enabling faster and more accurate decision-making.

A hallmark feature of the system is the Digital Disease Activity Score (DDAS). This innovative scoring mechanism combines objective data regarding range-of-motion (ROM) with patient-reported outcomes, which include aspects such as functional capacity, pain intensity, and the severity of tenderness and swelling. By integrating these data points, the DDAS offers a holistic view of disease activity, closely aligning with DAS-28, a gold standard in clinical settings. The system uses pose estimation models to establish thresholds for healthy ROM, comparing them against patient data to identify deviations indicative of swelling or restricted motion. These clinically validated thresholds enable the system to execute accurate and nuanced assessments, empowering it to predict potential flare-ups, track disease progression, and provide dynamically tailored care recommendations based on individual patient profiles.

The system's user interface enhances its accessibility and utility. Designed with multilingual capabilities, the interface provides clear, concise instructions for patients conducting self-assessments while offering clinicians an analytics dashboard to review both real-time data and historical trends. This interface ensures that interactions are intuitive and efficient, improving the overall user experience and facilitating better data interpretation and clinical decision-making.

Cloud integration is another critical feature, ensuring that all data are stored, transferred, and processed securely. This facilitates seamless data sharing between patients and clinicians, supports real-time monitoring capabilities, and ensures compliance with stringent data protection regulations such as DPA, GDPR and HIPAA. By safeguarding patient information, the system maintains high standards of privacy and security, which are paramount in the healthcare setting.

Beyond standard capabilities, the system is equipped with advanced features, including flare-up reporting, progress tracking, and integration with electronic health records (EHR) systems, enhancing the efficiency of clinical workflows. The adaptable AI architecture of the platform can accommodate new data inputs, allowing for continuous improvement and customisation. This feature ensures that the system remains at the forefront of technological advancements and evolving clinical needs, offering scalable solutions that can be tailored to individual patients or broader clinical applications.

In summary, the AI-powered system for monitoring joint health introduces a transformative approach to arthritis care by replacing traditional, subjective assessments with precise, AI-driven analytics. Unlike conventional methods that depend on manual measurements and in-person evaluations, this system leverages advanced algorithms to deliver real-time insights, integrate patient-reported outcomes, and enable personalised care strategies, setting a new standard for efficiency and accuracy in arthritis management. By combining real-time motion tracking with patient-reported data and advanced analytics, the system uniquely integrates objective measurements with subjective patient inputs, creating a comprehensive evaluation of disease activity. This integration ensures an accurate depiction of the patient's condition, facilitating personalised treatment strategies and evidence-based clinical decisions. This innovative system not only improves patient outcomes through personalised care but also alleviates healthcare burdens by reducing unnecessary in-person evaluations and enhancing cost efficiency. Ultimately, it positions itself as an indispensable tool in the modern management and treatment of arthritis by offering unparalleled scalability, cost efficiency, and the ability to personalise care. These features not only optimise clinical workflows but also empower patients and clinicians with real-time, actionable insights, transforming arthritis care into a more precise and effective process.

1. A method for remotely capturing and analysing joint positions using video data or static images obtained from a camera-equipped device, including but not limited to smartphones, tablets, IoT devices, wearable devices, or any future camera equipped apparatus capable of capturing joint movements, joint swelling or discolouration, wherein the method comprises: guiding patients through predefined exercises for data consistency; extracting video frames or static images; and processing the data through AI-driven algorithms optimised for clinical precision, assessing joint range of motion and detecting indicators of arthritic conditions.

1.1. The method of claim 1, further comprising the detection of skin discoloration and swelling through the analysis of visual markers in video frames or static images.

1.2. The method of claim 1, wherein the extracted data is compared against clinically established predefined static thresholds derived from extensive clinical measurements, to identify deviations from healthy joint ranges, thereby enabling precise and reliable assessments of disease activity.

1.3. The method of claim 1, wherein the AI-driven algorithms, developed in Python, are trained on extensive, clinically validated datasets to ensure high accuracy and reliability in joint health analysis, leveraging advanced techniques to consistently deliver precise assessments.

1.4. The method of claim 1, wherein the AI-driven algorithms comprise convolutional neural networks (CNNs), and random forest models trained on datasets of annotated extracted joint data, images and videos of joints, wherein the datasets are continuously updated with new patient data to improve model accuracy and adaptability.

1.5. The method of claim 1, wherein the AI-driven algorithms are trained using supervised learning techniques, with labelled data, to detect joint positions, angles, range of motion (ROM), and visual indicators of arthritis, including swelling and skin discoloration.

1.6. The method of claim 1, wherein the AI-driven algorithms are configured to perform real-time processing of video and image data locally (when used through the downloaded app) on the camera-equipped device using edge computing techniques, enabling low-latency analysis of joint movements and immediate feedback to patients and clinicians. Alternatively, when accessed via a web browser, the system securely transmits data to cloud servers for processing, ensuring scalability and consistent performance across platforms.

1.7. The method of claim 1, wherein the system employs edge computing to process video and image data locally on the device, reducing the need for cloud-based processing and ensuring efficient analysis in low-bandwidth environments.

2. An integrated system for evaluating joint health and disease severity in arthritis patients, comprising: a Video Capture Module for recording joint movements; an AI Analysis Engine for processing captured data; and a Digital Disease Activity Score (DDAS) mechanism that synthesises objective range of motion data with patient-reported outcomes, generating comprehensive disease activity scores.

2.1. The system of claim 2, further comprising a user interface that provides multilingual, intuitive instructions for patient assessments and offers clinicians analytics for reviewing assessment data.

2.2. The system of claim 2, wherein data handling is enhanced through secure cloud integration, ensuring compliance with DPA, GDPR and HIPAA for safe data storage, transfer, and processing. All data is encrypted both in transit (via HTTPS) and at rest, ensuring that it cannot be intercepted or accessed by unauthorised users during transmission or storage.

2.3. The system of claim 2, wherein the DDAS mechanism incorporates weighted computations aligned with clinical standards for a nuanced analysis of disease activity. These computations combine objective data from joint range of motion and patient-reported indicators, enabling a holistic evaluation of arthritis severity. By integrating thresholds established through validated pose estimation models, the system ensures precise and reliable assessments that directly inform personalised treatment plans and clinical decision-making.

2.4. The system of claim 2, further comprising advanced features such as flare-up reporting, progress tracking, and integration with electronic health record (EHR) systems for streamlined clinical workflows.
